(19) United States
(12) Patent Application Publication
(10) Pub. No.: US 2022/0237775 A1
(43) Pub. Date: Jul. 28, 2022

(54) RADIOGRAPHIC IMAGING SYSTEM, RADIOGRAPHIC IMAGING APPARATUS, AND INSPECTION METHOD FOR INSPECTING RADIOGRAPHIC IMAGING APPARATUS

(71) Applicant: CANON KABUSHIKI KAISHA, Tokyo (JP)

(72) Inventor: Takashi Takasaki, Kanagawa (JP)

(21) Appl. No.: 17/581,398

(22) Filed: Jan. 21, 2022

(30) Foreign Application Priority Data
Jan. 27, 2021 (JP) 2021-011301

(51) Int. Cl. G06T 7/00; G06V 10/22; G06T 7/70; H04N 5/32; G06T 7/11; G06T 7/68; A61B 6/00

(52) U.S. Cl. CPC G06T 7/0012; G06V 10/22; G06T 7/70; H04N 5/32; G06T 7/11; G06T 2207/30004; A61B 6/4208; G06V 2201/03; G06T 2207/20021; G06T 2207/10116; G06T 7/68

(57) ABSTRACT

A radiographic imaging system including a radiographic imaging apparatus having a pixel array including a dose detection region where dose detection pixels for outputting signals to be used to detect a dose of emitted radiation is provided, based on position information regarding positions in the dose detection region of defective pixels among the dose detection pixels, determines whether radiographic imaging involving the detection of the dose of the radiation can be normally performed.

### BACKGROUND

### Field of the Disclosure

The present disclosure relates to a radiographic imaging system, a radiographic imaging apparatus, and an inspection method for inspecting a radiographic imaging apparatus.

### Description of the Related Art

Currently, as an imaging apparatus used in medical diagnostic imaging or nondestructive inspection with radiation, a radiographic imaging apparatus using a flat-panel detector (hereinafter “FPD”) formed of a semiconductor material is prevalent. For example, in medical diagnostic imaging, such a radiographic imaging apparatus is used as a digital imaging apparatus capable of performing still image capturing such as general image capturing and moving image capturing such as fluoroscopic image capturing in a radiographic imaging system.

The radiographic imaging system has the function of monitoring the irradiation dose of radiation, and in a case where the irradiation dose reaches a target value, ending the emission of the radiation (e.g., outputting a signal for stopping the emission of the radiation to a radiation source). This function is termed automatic exposure control (hereinafter “AEC”), and for example, can prevent the excessive emission of radiation.

Japanese Patent Application Laid-Open No. 2019-146039 discusses a radiographic imaging apparatus including, in a dose detection region in a pixel array in which pixels for outputting image signals according to radiation (image output pixels) are disposed in a two-dimensional matrix, pixels for detecting the irradiation dose of the radiation (dose detection pixels).

Japanese Patent Application Laid-Open No. 2019-146039 discusses a technique for monitoring the irradiation dose of the radiation by adding signals of dose detection pixels placed in a plurality of rows in the dose detection region in the pixel array. To improve the accuracy of correction of a defective pixel present in the dose detection region using pixels near the defective pixel, Japanese Patent Application Laid-Open No. 2019-146039 also discusses a technique for disposing at least one row of image output pixels between the defective pixel and a row in which dose detection pixels are disposed.

In the technique of Japanese Patent Application Laid-Open No. 2019-146039, in a case where a plurality of defective pixels occurs when an FPD is manufactured, a minimum of one row of image output pixels may not be able to be placed between two rows in which dose detection pixels are placed. If such an FPD is used, a defective pixel is not corrected with high accuracy, and the grade of an image decreases. Thus, the FPD cannot normally perform radiographic imaging involving an AEC function.

Thus, when an FPD is manufactured, then based on the state of a defective pixel in the dose detection region, it is determined whether the FPD can normally perform radiographic imaging involving the AEC function. If a criterion for normality is set too strictly when the determination is made, normality is determined as abnormality, whereby the yield in the manufacturing of the FPD decreases.

### SUMMARY OF THE INVENTION

According to an aspect of the present invention, a method for appropriately determining whether the FPD can normally perform radiographic imaging involving the AEC function is provided, and the above issue is solved by a radiographic imaging system including a radiographic imaging apparatus having a pixel array, the pixel array including a dose detection region where dose detection pixels for outputting signals to be used to detect a dose of emitted radiation is provided, the radiographic imaging system including a determination unit configured to, based on position information regarding positions in the dose detection region of at least either normal pixels or defective pixels among the dose detection pixels, determine whether radiographic imaging involving the detection of the dose of the radiation can be normally performed.

Further features of the present invention will become apparent from the following description of exemplary embodiments with reference to the attached drawings.

### BRIEF DESCRIPTION OF THE DRAWINGS

FIG. 1 is a diagram illustrating a configuration of a radiographic imaging system according to a first exemplary embodiment.

FIG. 2 is a diagram illustrating a configuration of a radiographic imaging apparatus according to the first exemplary embodiment.

FIG. 3 is a flow illustrating processing from extraction of defective pixels to determination according to the first exemplary embodiment.

FIG. 4 is a flow for determining the radiographic imaging apparatus according to the first exemplary embodiment.

FIGS. 5A and 5B are diagrams illustrating a method for determining whether center coordinates of normal pixels included in dose detection pixels satisfy a criterion according to the first exemplary embodiment.

FIGS. 6A and 6B are diagrams illustrating a method for determining whether the number of defective pixels included in the dose detection pixels satisfies a criterion with respect to each sub-region according to the first exemplary embodiment.

FIG. 7 is a flow illustrating processing from a start to an end of imaging of an object according to the first exemplary embodiment.

### DESCRIPTION OF THE EMBODIMENTS

With reference to the attached drawings, suitable exemplary embodiments to which the present invention is applied will be described in detail below.

In a radiographic imaging system according to the present invention, typically, radiation can be an X-ray. The radiographic imaging system according to the present invention, however, can also be applied to a radiographic imaging system using not only an X-ray but also another type of radiation (e.g., an α-ray, a β-ray, or a γ-ray).

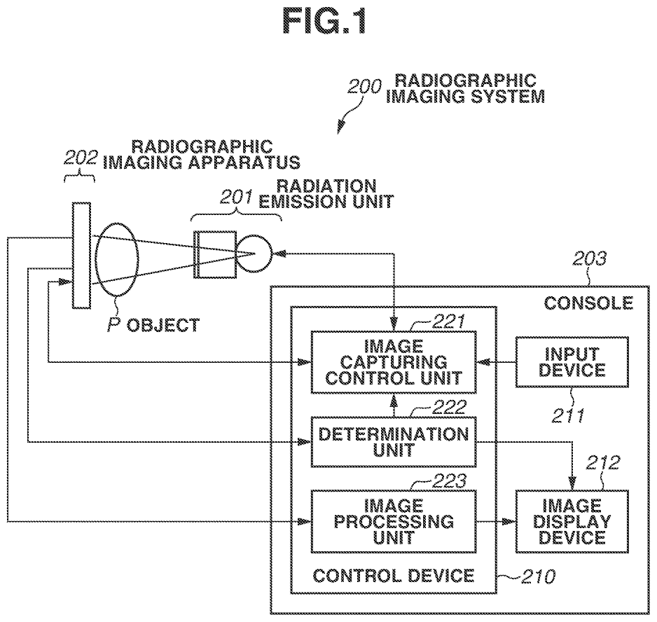

First, a first exemplary embodiment of the present invention is described. FIG. 1 is a diagram illustrating the overall configuration of a radiographic imaging system 200 according to the first exemplary embodiment of the present invention. The radiographic imaging system 200 according to the present invention is particularly used in medical applications. The radiographic imaging system 200 includes a radiation emission unit 201, a radiographic imaging apparatus 202, and a console 203.

The radiation emission unit 201 emits radiation to an object P. The radiation emission unit 201 includes a radiation generation unit (an X-ray tube) as a radiation generation device, a collimator that defines the beam spread angle of radiation generated by the radiation generation unit, and a radiation dose measuring device attached to the collimator.

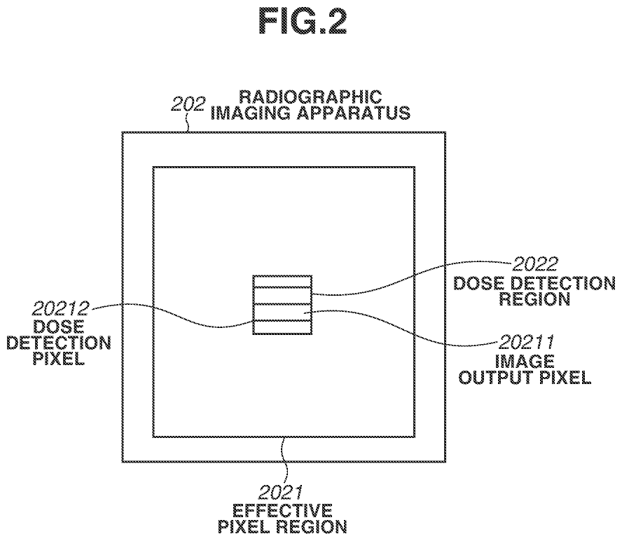

The radiographic imaging apparatus 202 is a flat-panel detector (FPD) and includes two-dimensionally distributed image sensors. The details of the configuration of the radiographic imaging apparatus 202 will be described below with reference to FIG. 2. The radiographic imaging apparatus 202 detects the two-dimensional distribution of radiation having reached the radiographic imaging apparatus 202 and generates radiographic image data. The radiographic imaging apparatus 202 transmits the generated radiographic image data to an image processing unit 223. The radiographic imaging apparatus 202 also transmits information regarding the dose of the detected radiation to an image capturing control unit 221 and a determination unit 222. The details of the image capturing control unit 221, the determination unit 222, and the image processing unit 223 will be described below.

The console 203 includes a control device 210, an input device 211, and an image display device 212. As the form of the console 203, a general-purpose computer may be used, or the console 203 may be provided as an operation panel dedicated to the radiographic imaging system 200.

The control device 210 is a device for controlling the radiographic imaging system 200. As the control device 210, a central processing unit (CPU) in a computer is used. The control device 210 includes the image capturing control unit 221, the determination unit 222, and the image processing unit 223. As the input device 211, a keyboard, a mouse, and a touch panel are used, and an operator inputs imaging conditions such as an image capturing part, a target dose of radiation to be emitted to the object P, and a tube voltage. The input imaging conditions are transmitted to the image capturing control unit 221.

The image display device 212 displays a captured radiographic image and a screen for inputting imaging conditions. As the image display device 212, a general-purpose display is used. Alternatively, the image display device 212 may function also as the input device 211 using a touch panel.

Based on information regarding imaging conditions transmitted from the input device 211 and information regarding the dose of radiation received from the radiographic imaging apparatus 202, the image capturing control unit 221 controls the radiation emission unit 201 and the radiographic imaging apparatus 202.

Based on information regarding the dose of radiation transmitted from the radiographic imaging apparatus 202, the determination unit 222 determines whether each pixel in the radiographic imaging apparatus 202 is a normal pixel or a defective pixel. Further, based on the result of determining whether each pixel in the radiographic imaging apparatus 202 is a normal pixel or a defective pixel, the determination unit 222 determines whether the radiographic imaging apparatus 202 can normally detect the irradiation dose. The details of the determination will be described below. The determination unit 222 is provided in the console 203 in the present exemplary embodiment, but may be provided in the radiographic imaging apparatus 202. In this case, the function of the determination unit 222 may be achieved using a CPU provided in the radiographic imaging apparatus 202.

The image processing unit 223 performs processes such as a gradation process and a noise reduction process on radiographic image data transmitted from the radiographic imaging apparatus 202. The image processing unit 223 transmits the processed radiographic image data to the image display device 212. The image display device 212 outputs image information transmitted from the image processing unit 223.

With reference to FIG. 2, the configuration of the radiographic imaging apparatus 202 is described. The radiographic imaging apparatus 202 includes a pixel array in which image output pixels 20211 and dose detection pixels 20212 are arranged in a two-dimensional array. An effective pixel region 2021 in the radiographic imaging apparatus 202 includes the image output pixels 20211. The image output pixels 20211 are pixels that detect radiation having reached the radiographic imaging apparatus 202 and generate charges according to the radiation to generate radiographic image data.

The effective pixel region 2021 also includes a dose detection region 2022 that detects the irradiation dose of radiation. The dose detection region 2022 includes the image output pixels 20211 and the dose detection pixels 20212 that are pixels for detecting the irradiation dose of emitted radiation. The dose detection pixels 20212 in the dose detection region 2022 are formed in three rows.

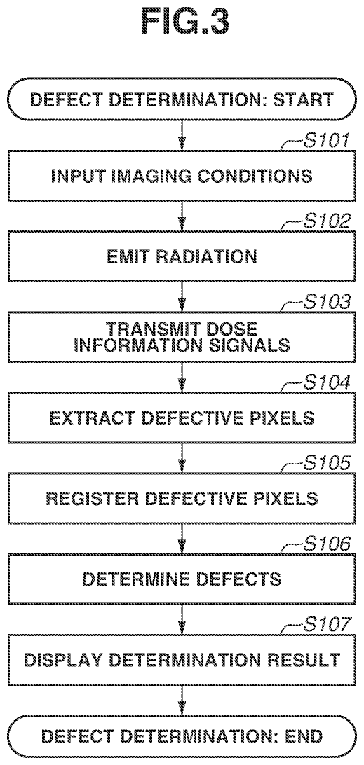

With reference to FIG. 3, the flow of an inspection for determining the radiographic imaging apparatus 202 according to the first exemplary embodiment of the present invention is illustrated below. In the first exemplary embodiment, the processing of this inspection flow is performed in the step of manufacturing the radiographic imaging apparatus 202.

In step S101, the operator inputs a tube voltage kV, a tube current mA, and an irradiation time ms as imaging conditions using the input device 211. The input imaging conditions are transmitted to the image capturing control unit 221.

In step S102, based on the information regarding the received imaging conditions, the image capturing control unit 221 controls the radiation emission unit 201 to emit radiation under the conditions of the tube voltage kV, the tube current mA, and the irradiation time ms.

Then, the image capturing control unit 221 transmits an image capturing control signal to the radiographic imaging apparatus 202. Then, based on the received image capturing control signal, the radiographic imaging apparatus 202 controls the image output pixels 20211 and the dose detection pixels 20212 to convert radiation having reached the radiographic imaging apparatus 202 into dose information signals with respect to each pixel.

In step S103, the radiographic imaging apparatus 202 transmits the dose information signals with respect to each pixel of the dose detection pixels 20212 to the determination unit 222.

In step S104, the determination unit 222 determines whether the dose information signals with respect to each pixel of the dose detection pixels 20212 received in step S103 are greater than or equal to a normal pixel determination threshold Smin determined in advance and is less than or equal to a normal pixel determination threshold Smax determined in advance. The determination unit 222 determines as a defective pixel a pixel that is not a pixel that is greater than or equal to the normal pixel determination threshold Smin and is less than or equal to the normal pixel determination threshold Smax.

In step S105, the determination unit 222 saves coordinate information regarding the pixels determined as the defective pixels in step S104 within the determination unit 222.

In step S106, the determination unit 222 determines whether the defective pixels extracted in step S104 are in an acceptable range where the radiographic imaging apparatus 202 detects the irradiation dose. The details of the determination will be described below.

In step S107, the determination unit 222 transmits information regarding the determination result to the image display device 212. The image display device 212 displays the information regarding the determination result to the operator. Based on the displayed result, the operator determines whether to capture the object P. By the above processing, the flow from the extraction of defective pixels to the quality determination is completed.

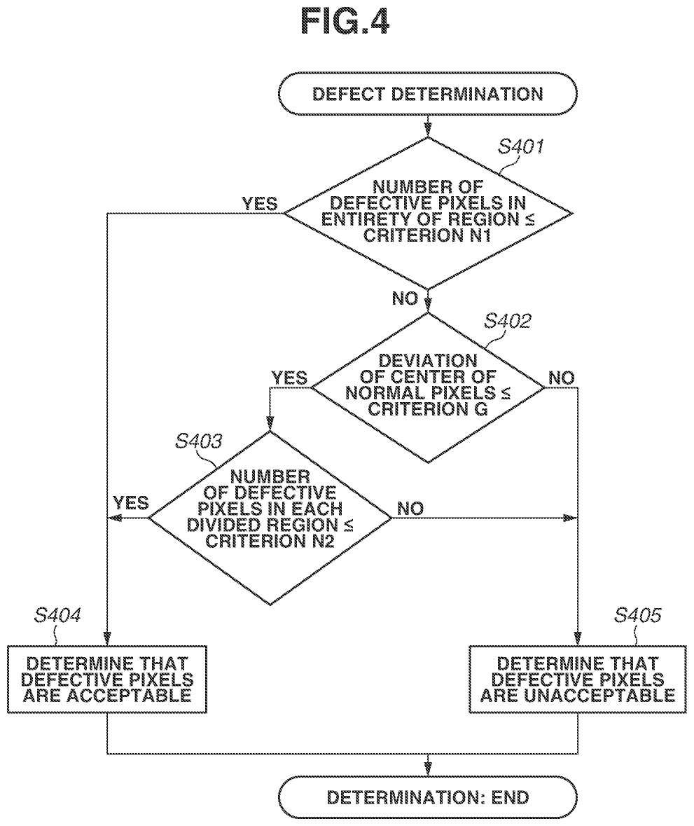

With reference to FIG. 4, the flow of the determination made by the determination unit 222 in step S106 is described below.

In step S401, if the total number of the defective pixels counted in step S104 is less than or equal to a criterion N1 as a criterion value (Yes in step S401), the determination unit 222 determines that the defective pixels are acceptable. Then, the processing proceeds to step S404. If, on the other hand, the total number of the defective pixels exceeds the criterion N1 (No in step S401), the processing proceeds to step S402. In step S402, the image capturing control unit 221 determines whether the deviation of the center coordinates of normal pixels included in the dose detection pixels 20212 in the dose detection region 2022 is less than or equal to a criterion G. This determination method will be described below. If it is determined in step S402 that the deviation is less than or equal to the criterion G (Yes in step S402), the processing proceeds to step S403 in FIG. 4.

In step S403, with respect to each of regions obtained by dividing the dose detection region 2022, the determination unit 222 counts the number of defective pixels included in the dose detection pixels 20212 and determines whether the number of defective pixels included in the dose detection pixels 20212 is less than or equal to a criterion N2 as a criterion value. Then, the determination unit 222 determines whether the defective pixels are acceptable. If the defective pixels are acceptable (Yes in step S403), the processing proceeds to step S404. If the defective pixels are unacceptable (No in step S403), the processing proceeds to step S405. In steps S404 and S405, it is determined that the defective pixels are either acceptable or unacceptable. Then, the flow in FIG. 4 ends.

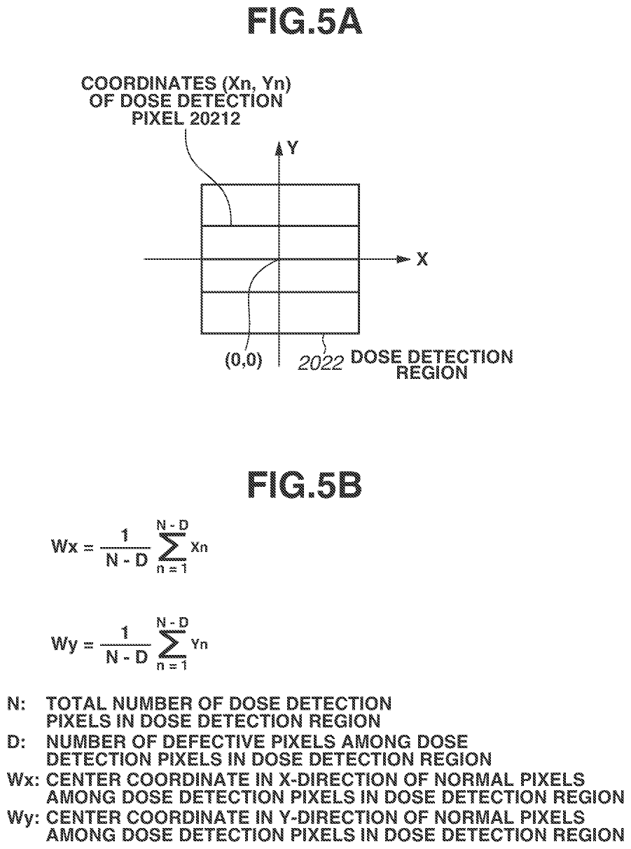

With reference to FIGS. 5A and 5B, a specific method for the determination made by the determination unit 222 in step S402 is described below.

First, as illustrated in FIG. 5A, the determination unit 222 sets X-coordinates and Y-coordinates in the dose detection region 2022. The center of the X-coordinates and the Y-coordinates is matched to the center of the dose detection region 2022. Then, the image capturing control unit 221 extracts coordinates (Xn, Yn) of normal dose detection pixels 20212 included in the dose detection region 2022. Then, using formulas illustrated in FIG. 5B, the determination unit 222 calculates center coordinates (Wx, Wy) of the normal dose detection pixels 20212 included in the dose detection region 2022.

Then, the determination unit 222 determines whether the calculated center coordinates Wx and Wy are both less than or equal to the criterion G as a criterion value for the deviation of the center. If it is determined that the center coordinates Wx and Wy are greater than the criterion G, the determination unit 222 determines that the defective pixels are unacceptable. The reason for the determination that the defective pixels are unacceptable is that the center of the dose detection region 2022 and the center of dose detection pixels 20212 used to detect the dose are shifted from each other, whereby the dose cannot be normally detected. If the dose cannot be normally detected, the dose value of radiation to be actually emitted to the radiographic imaging apparatus 202 is highly likely to exceed a clinically acceptable value.

If it is determined that the center coordinates Wx and Wy are less than or equal to the criterion G, the processing proceeds to step S403 in the flow in FIG. 4.

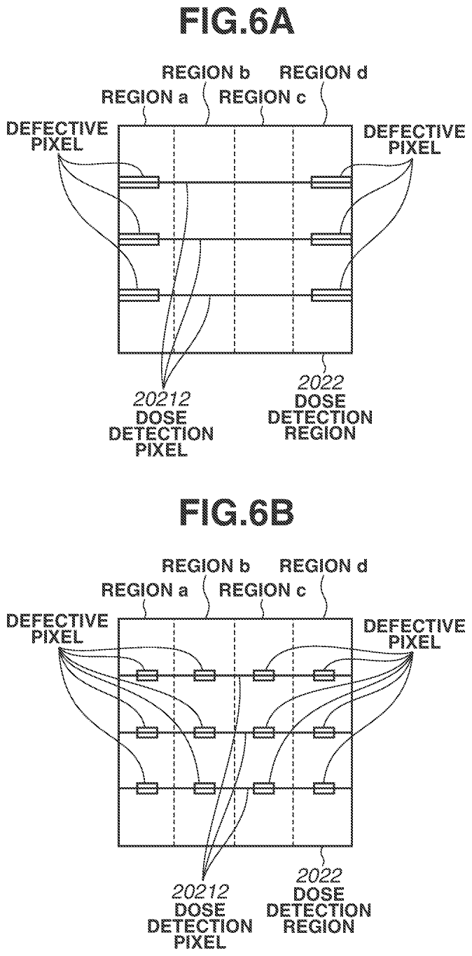

With reference to FIGS. 6A and 6B, a specific method for the determination made in step S403 is described below.

As illustrated in FIGS. 6A and 6B, the determination unit 222 further divides the dose detection region 2022 into sub-regions, namely regions a, b, c, and d. With respect to each of the regions a, b, c, and d, the determination unit 222 determines whether the number of defective pixels included in the dose detection pixels 20212 is less than or equal to the criterion N2.

If the number of defective pixels included in the dose detection pixels 20212 is less than or equal to the criterion N2 in all of the regions a, b, c, and d, the processing proceeds to step S404 in the flow in FIG. 4. In step S404, the determination unit 222 that determines that the defective pixels are acceptable. If the number of defective pixels included in the dose detection pixels 20212 exceeds the criterion N2 in any one of the regions a, b, c, and d, the processing proceeds to step S405 in the flow in FIG. 4. In step S405, the determination unit 222 determines that the defective pixels are unacceptable.

The reason for the determination that the defective pixels are unacceptable is that if defective pixels concentrate in a particular region, irradiation dose information regarding the particular region cannot be detected, and the dose cannot be normally detected. If the dose cannot be normally detected, the dose value of radiation to be emitted to the radiographic imaging apparatus 202 is highly likely to exceed a clinically acceptable value.

In the example illustrated in FIG. 6A, the number of defective pixels included in the dose detection pixels 20212 exceeds the criterion N2 in the regions a and d. Thus, the determination unit 222 determines that the defective pixels are unacceptable. In the example illustrated in FIG. 6B, the number of defective pixels included in the dose detection pixels 20212 is less than or equal to the criterion N2 in all of the regions a, b, c, and d. Thus, the determination unit 222 determines that the defective pixels are acceptable. By the above processing, it can be determined whether the defective pixels extracted in step S104 are in the acceptable range where the radiographic imaging apparatus 202 detects the irradiation dose.

That is, in the present invention, the determination unit 222 as a determination unit determines whether the dose of radiation can be normally detected. The determination is made based on position information regarding the positions in the dose detection region 2022 of at least either normal pixels or defective pixels among the dose detection pixels 20212.

The position information regarding the positions in the dose detection region 2022 is, as illustrated in FIG. 5A, information regarding the positions of the normal pixels or the defective pixels relative to the dose detection region 2022 in the radiographic imaging apparatus 202 as a target of the determination. In the present exemplary embodiment, the position information regarding the positions in the dose detection region 2022 is information representing the positions where the normal pixels or the defective pixels are present in the dose detection region 2022, as XY-coordinates. The position information also includes values calculated based on the XY-coordinates.

A method for the determination based on the position information is performed by, for example, obtaining the deviation between the geometric center of the dose detection region 2022 and the geometric center of the normal pixels and comparing the deviation with a criterion value determined in advance. Alternatively, the method for the determination is also performed by comparing the number of defective pixels included in each of the sub-regions obtained by further dividing the dose detection region 2022 with a criterion value determined in advance. Yet alternatively, the method for the determination may be a method other than the above determination methods so long as the method is based on information regarding the positions of the normal pixels or the defective pixels relative to the dose detection region 2022.

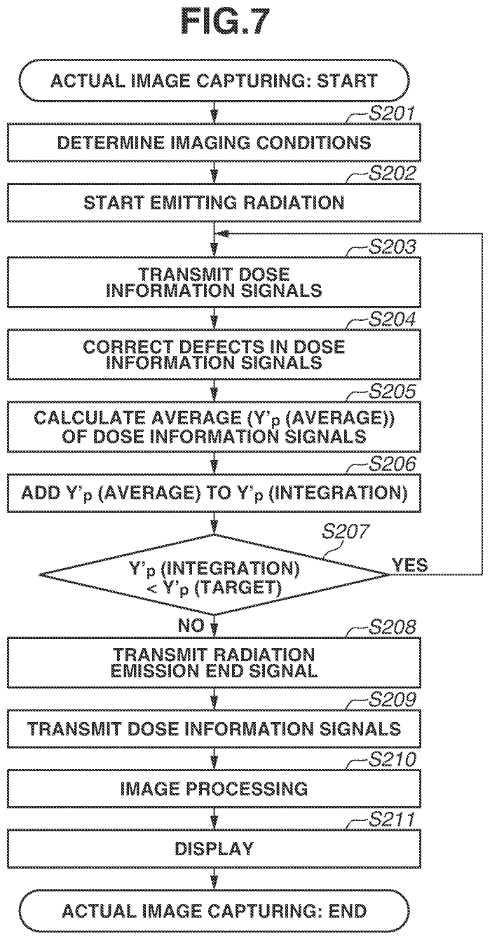

With reference to FIG. 7, the flow from the start to the end of the imaging of an object according to the first exemplary embodiment of the present invention is illustrated below.

In step S201, the operator inputs the targets of the tube voltage kV, the tube current mA, and a target dose Yp using the input device 211. The input imaging conditions are transmitted to the image capturing control unit 221.

In step S202, based on the information regarding the received imaging conditions, the image capturing control unit 221 controls the radiation emission unit 201 to emit radiation to the object P under the conditions of the tube voltage kV and the tube current mA. Then, the image capturing control unit 221 transmits an object image capturing control signal to the radiographic imaging apparatus 202. Then, based on the received image capturing control signal, the radiographic imaging apparatus 202 controls the image output pixels 20211 and the dose detection pixels 20212 to convert radiation having reached the radiographic imaging apparatus 202 into dose information signals Y′p, m, and n with respect to each pixel. At this time, the dose detection pixels 20212 are driven in a shorter accumulation time than the image output pixels 20211 and at a higher frame rate than the image output pixels 20211.

In step S203, the radiographic imaging apparatus 202 transmits the dose information signals Y′p, m, and n with respect to each pixel of the dose detection pixels 20212 to the image capturing control unit 221. Y′p is the value of the dose, and m and n are the X-coordinate and the Y-coordinate, respectively, illustrated in FIG. 5A.

In step S204, the image capturing control unit 221 performs a defect correction process on the dose information signals Y′p, m, and n with respect to each pixel of the dose detection pixels 20212 received in step S203. Signals output from defective pixels cannot be used for image data, and therefore are excluded. Thus, the image capturing control unit 221 excludes signals of pixels corresponding to the coordinates of the defective pixels among the dose detection pixels 20212 saved in step S104 from the dose information signals Y′p, m, and n with respect to each pixel of the dose detection pixels 20212 received in step S203. Generally, a portion where each defective pixel is excluded is corrected using information regarding pixels around the defective pixel.

For example, the average value of signals of eight pixels around the defective pixel may be the output value of the portion where the defective pixel has been present. Alternatively, for example, a value calculated by appropriately weighting the signals of the eight pixels around the defective pixel may be the output value of the portion where the defective pixel has been present. The correction of the defective pixel is not limited to the correction using the eight pixels around the defective pixel, and the number of pixels and a region to be used may be variable.

In step S205, the image capturing control unit 221 calculates an average value Y′p (average) of all pixel signals of the dose information signals Y′p, m, and n with respect to each pixel of the dose detection pixels 20212, except the signals excluded in step S204.

In step S206, the image capturing control unit 221 adds Y′p (average) calculated in step S205 to Y′p (integration) that is information regarding the dose of radiation having reached the radiographic imaging apparatus 202 after having passed through the object P. The initial value of Y′p (integration) is 0.

In step S207, the image capturing control unit 221 determines whether Y′p (integration) calculated in step S206 is less than Y′p (target) that is the target dose received in step S201. If it is determined that Y′p (integration) is less than the target dose (Yes in step S207), the processes of steps S203 to S206 are repeated. If it is determined that Y′p (integration) is greater than or equal to Y′p (target) (No in step S207), the processing proceeds to step S208.

In step S208, the image capturing control unit 221 transmits a radiation emission end signal to the radiation emission unit 201. Based on the received signal, the radiation emission unit 201 stops emitting the radiation. Then, the image capturing control unit 221 transmits an image capturing control signal to the radiographic imaging apparatus 202. Then, based on the received image capturing control signal, the radiographic imaging apparatus 202 controls the image output pixels 20211 and the dose detection pixels 20212 to end the conversion of radiation into the dose information signals.

In step S209, the radiographic imaging apparatus 202 transmits dose information signals Yp, i, and j with respect to each pixel of the image output pixels 20211 after the conversion ends in step S208 to the image processing unit 223. Yp is the value of the dose, and i and j are the X-coordinate and the Y-coordinate, respectively, illustrated in FIG. 5A similarly to m and n.

In step S210, the image processing unit 223 performs a gradation process and a noise reduction process on the dose information signals Yp, i, and j with respect to each pixel of the image output pixels 20211 received in step S209. Next, the image processing unit 223 transmits the processed signals to the image display device 212.

Next, in step S211, the image display device 212 converts received information into a two-dimensional image and displays the two-dimensional image to the operator. Based on the above, the processing of the image capturing of an object ends.

In the first exemplary embodiment, the processing from the extraction of defective pixels to the quality determination may be performed when the radiographic imaging system 200 is installed at a use location. In the first exemplary embodiment, the processing from the extraction of defective pixels to the quality determination may be performed periodically such as every month.

In the first exemplary embodiment, in step S106, the determination may be made using the dispersion or the standard deviation of the coordinates of the defective pixels included in the dose detection pixels 20212 in the dose detection region 2022. In this case, if the calculated dispersion or standard deviation is less than a criterion value, i.e., if the defective pixels are dense, it may be determined that the radiographic imaging apparatus 202 is a defective product. Similarly, the determination may be made using the dispersion or the standard deviation of the coordinates of the normal pixels included in the dose detection pixels 20212.

The purpose of the determination is as follows. In a case where the defective pixels are dense in a particular portion in the dose detection region 2022, and even if the geometric center of the normal pixels is close to the center of the dose detection region 2022, the dose value in the dose detection region 2022 cannot be normally detected. If the dose value cannot be normally detected, the dose value of radiation to be actually emitted to the radiographic imaging apparatus 202 is highly likely to exceed a clinically acceptable value.

In the first exemplary embodiment, the dose detection region 2022 is divided into four vertical regions, namely the regions a, b, c, and d. Alternatively, the number of divisions may be changed, and the number of defective pixels may be determined in each region. Alternatively, the direction of divisions may be changed to the horizontal direction or both the vertical and horizontal directions, and the number of defective pixels may be determined in each divided region.

In the first exemplary embodiment, in the situation where a determination is made using defective pixels, the determination may be made using normal pixels. For example, in both the determination made by counting the number of defective pixels in step S401 and the determination made by counting defective pixels in each divided sub-region in step S403, the determinations may be made by providing criterion values for the numbers of normal pixels and counting normal pixels. Similarly, in the situation where a determination is made using normal pixels, the determination may be made using defective pixels. For example, in step S402, the deviation between the coordinates of the geometric center of the normal pixels in the dose detection region 2022 and the center coordinates of the dose detection region 2022 is obtained. Alternatively, the coordinates of the geometric center of the defective pixels may be used.

Only any one of the determination methods described in the first exemplary embodiment, i.e., the determination method in step S402, the determination method in step S403, and the determination method using the dispersion or the standard deviation of the coordinates of the defective pixels or the normal pixels, may be used.

By the above inspection, it can be appropriately determined whether a radiographic imaging apparatus having an automatic exposure control (AEC) function can normally perform radiographic imaging involving the AEC function.

### Other Exemplary Embodiments

The present invention can also be achieved by the process of supplying a program for achieving the above functions to a system or an apparatus via a network or a storage medium, and of causing one or more processors of a computer of the system or the apparatus to read and execute the program.

As the storage medium, various storage media such as a flexible disk, an optical disc (e.g., a Compact Disc Read-Only Memory (CD-ROM) or a Digital Versatile Disc Read-Only Memory (DVD-ROM)), a magneto-optical disc, a magnetic tape, a non-volatile memory (e.g., a Universal Serial Bus (USB) memory), and a read-only memory (ROM) can be used. The program for achieving the above functions may be downloaded via the network and executed by the computer.

The present invention is not limited to a case where the functions of the above exemplary embodiments are achieved by executing a program code read by the computer. The present invention also includes a case where based on an instruction from the program code, an operating system (OS) operating on the computer performs a part or all of actual processing, and the functions of the above exemplary embodiments are achieved by the processing.

Further, the program code read from the storage medium may be written to a memory included in a function extension board inserted into the computer or a function extension unit connected to the computer. The present invention also includes a case where based on an instruction from the program code, a CPU included in the function extension board or the function extension unit performs a part or all of actual processing, and the above functions are achieved by the processing.

According to the present invention, a radiographic imaging system that appropriately determines whether a radiographic imaging apparatus having an AEC function can normally perform radiographic imaging involving the AEC function is provided.

### Other Embodiment

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)?), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-011301, filed Jan. 27, 2021, which is hereby incorporated by reference herein in its entirety.

What is claimed is:

1. A radiographic imaging system including a radiographic imaging apparatus having a pixel array, the pixel array including a dose detection region where a plurality of dose detection pixels for outputting signals to be used to detect a dose of emitted radiation is provided, the radiographic imaging system comprising:
a determination unit configured to, based on position information regarding positions in the dose detection region of at least either one or more normal pixels or one or more defective pixels among the plurality of dose detection pixels, determine whether radiographic imaging involving the detection of the dose of the radiation can be normally performed.

2. The radiographic imaging system according to claim 1,
wherein the position information is a deviation between a geometric center of the dose detection region and a geometric center of the one or more normal pixels in the dose detection region, and
wherein the determination unit makes the determination by comparing the deviation and a criterion value determined in advance.

3. The radiographic imaging system according to claim 1,
wherein the position information is a deviation between a geometric center of the dose detection region and a geometric center of the one or more defective pixels in the dose detection region, and
wherein the determination unit makes the determination by comparing the deviation and a criterion value determined in advance.

4. The radiographic imaging system according to claim 1,
wherein the dose detection region includes a plurality of sub-regions obtained by further dividing the dose detection region,
wherein the position information is the number of the one or more normal pixels included in each of the plurality of sub-regions, and
wherein the determination unit makes the determination by comparing the number of the one or more normal pixels and a criterion value determined in advance.

5. The radiographic imaging system according to claim 1,
wherein the dose detection region includes a plurality of sub-regions obtained by further dividing the dose detection region,
wherein the position information is the number of the one or more defective pixels included in each of the plurality of sub-regions, and
wherein the determination unit makes the determination by comparing the number of the one or more defective pixels and a criterion value determined in advance.

6. The radiographic imaging system according to claim 1,
wherein the dose detection region includes a plurality of sub-regions obtained by further dividing the dose detection region,
wherein the position information is a dispersion or a standard deviation of coordinates of the one or more normal pixels included in each of the plurality of sub-regions, and
wherein the determination unit makes the determination by comparing the dispersion or the standard deviation and a criterion value set in advance.

7. The radiographic imaging system according to claim 1,
wherein the dose detection region includes a plurality of sub-regions obtained by further dividing the dose detection region,
wherein the position information is a dispersion or a standard deviation of coordinates of the one or more defective pixels included in each of the plurality of sub-regions, and
wherein the determination unit makes the determination by comparing the dispersion or the standard deviation and a criterion value set in advance.

8. The radiographic imaging system according to claim 1, wherein the pixel array further includes image output pixels for generating charges according to the radiation to generate radiographic image data, and the dose detection pixels and the image output pixels are arranged in an array.

9. A radiographic imaging apparatus having a pixel array, the pixel array including a dose detection region where a plurality of dose detection pixels for outputting signals to be used to detect a dose of emitted radiation is provided, the radiographic imaging apparatus comprising:
a determination unit configured to, based on position information regarding positions in the dose detection region of at least either one or more normal pixels or one or more defective pixels among the plurality of dose detection pixels, determine whether radiographic imaging involving the detection of the dose of the radiation can be normally performed.

10. A control device that controls a radiographic imaging system including a radiographic imaging apparatus having a pixel array, the pixel array including a dose detection region where a plurality of dose detection pixels for outputting signals to be used to detect a dose of emitted radiation is provided, the control device comprising:
a determination unit configured to, based on position information regarding positions in the dose detection region of at least either one or more normal pixels or one or more defective pixels among the plurality of dose detection pixels, determine whether radiographic imaging involving the detection of the dose of the radiation can be normally performed.

11. An inspection method for inspecting a radiographic imaging apparatus having a pixel array, the pixel array including a dose detection region where a plurality of dose detection pixels for outputting signals to be used to detect a dose of emitted radiation is provided, the inspection method comprising:
based on position information regarding positions in the dose detection region of at least either one or more normal pixels or one or more defective pixels among the plurality of dose detection pixels, determining whether radiographic imaging involving the detection of the dose of the radiation can be normally performed.

12. A computer-readable storage medium storing a program for causing a computer to execute an inspection method for inspecting a radiographic imaging apparatus having a pixel array, the pixel array including a dose detection region where a plurality of dose detection pixels for outputting signals to be used to detect a dose of emitted radiation is provided, the inspection method comprising:
based on position information regarding positions in the dose detection region of at least either one or more normal pixels or one or more defective pixels among the plurality of dose detection pixels, determining whether radiographic imaging involving the detection of the dose of the radiation can be normally performed.
